(19) United States
(12) Patent Application Publication
(10) Pub. No.: US 2021/0137561 A1
(43) Pub. Date: May 13, 2021

(54) SYSTEM FOR REMOVING OBJECTS FROM THE THROAT

(71) Applicant: Manuel Munoz Saiz, Almeria (ES)

(72) Inventor: Manuel Munoz Saiz, Almeria (ES)

(21) Appl. No.: 17/091,207

(22) Filed: Nov. 6, 2020

(30) Foreign Application Priority Data
Nov. 7, 2019 (ES) 202000087

(51) Int. Cl. A61B 17/50

(52) U.S. Cl. CPC A61B 17/50; A61B 2017/00862

(57) ABSTRACT

The system for removing the objects from the throat consists of a device formed by a nozzle or a smooth, corrugated, waved or cylindrical tubular element, which is inserted into the mouth and allows a depression to be produced in it, either by extracting the tubular element smooth or cylindrical, or by sucking the air from the mouth with a corrugated tubular element by means of a suction pump constituted by a bellows, bell or chamber in the shape of a deformable spherical cap. The smooth or cylindrical tubular element is inserted into the mouth and once it is adjusted to the lips, it is extracted by sucking a large part of the air, creating a depression that sucks the object that closes the throat. The nozzle and the end of the corrugated tube are held in place with the teeth. Circulating the air through its central area. Valves with a flexible tongue act during the impulsion and extraction of air.

### INDEX TO RELATED APPLICATIONS

This application claims the benefit of Spanish application No. U201900549 filed Nov. 7, 2019, the disclosure of which is incorporated herein by reference in its entirety.

### FIELD OF THE INVENTION

In otorhinolaryngological disorders related to swallowing and breathing.

### STATE OF THE ART

In the market there are devices for treating throat obstruction with some solid food, and that due to lack of air in the lungs it is very difficult to extract them. Such devices are complex, difficult to use or handle, and not very useful or expensive. These inconveniences are solved with the present system.

### DESCRIPTION OF THE INVENTION

### Objective of the Invention and Advantages

To bring to the market simple devices, easy to handle or use, very useful and inexpensive.

To avoid choking for both the elderly and children.

To be able to save many lives.

To be able to use them anywhere, especially in dining rooms, schools, etc. each individual being able to carry his own.

When placed inside the mouth, you do not have to be aware of the opening of the same. The placement is simpler and more effective.

Useful for Cerebral palsy. Dysphagia. Asthma. Gastroesophageal reflux Muscular dystrophies. Huntington's disease. Brain damage. Parkinson. Dementia. Multiple sclerosis.

Amyotrophic lateral sclerosis (ALS). Intellectual and/or developmental disabilities Myasthenia gravis and drug intake neuroleptics and/or muscle relaxants. Esophagitis.

### Problem to Solve

The relative complexity, difficulty of adaptation and higher cost of the existing systems. Obstruction of the throat from food or objects stuck or trapped in the throat, including dense liquids, can lead to death from suffocation. Brain death can occur within four minutes. In Spain, a person chokes every 6 hours, 1,400 a year. It occurs both in healthy and sick people, children and especially in the elderly. Asphyxia by choking is one third of the causes of accidental death.

Swallowing is a complex process in which anatomical and physiological aspects are involved. This requires the coordination of voluntary movements (oral time) and involuntary movements (pharyngo-laryngo-esophageal time) (Arias, 1994). A large number of anatomical and muscular structures are involved in the act of swallowing. These structures have to function as an indissoluble unit.

The voluntary phase occurs once the food is ready for swallowing, the tongue compresses the bolus against the palate and voluntarily pushes it towards the pharynx, from this phase the others are involuntary. The involuntary or reflex phase is an automatic response made by a living being in the presence of a certain stimulus.

The oropharynx is the most complex pharyngeal subdivision. It is the regulator of the passage of air and food through the pharynx. It must have the ability to limit nasal pharyngeal reflux, propel the bolus, facilitate the passage of air and participate in phonation. The different muscle groups function as planes that enable or block some of the aero-digestive pathways.

The soft palate separates the rhinopharynx from the oropharynx. It is a quadrangular musculomembranous, sheet that describes four edges and two faces, upper rear and lower previous or rhinopharyngeal.

The pharyngeal phase begins with the flow of the food bolus from the base of the tongue to the rear pharyngeal wall. The soft palate rises to cover the nostrils and the oral cavity closes, the vocal cords contract causing the glottis to close, immediately the pharyngeal muscles retract causing movement of the bolus. The force caused by the movement of the bolus causes the epiglottis to cover the larynx so that food does not pass into the respiratory tract, then the upper esophageal sphincter relaxes allowing the bolus to enter the esophagus. This is the involuntary phase controlled by the swallowing centre in the medulla oblongata, since the nerve impulses to activate the reflexes of this process come directly from the swallowing centre to the efferents in the pharynx, these impulses travel through the nerve vagus to directly activate the required striated muscles, and there is no enteric nervous system intervention in these reflexes, since the pharyngeal muscles are striated.

The system for removing the objects from the throat, of the type that applies a suction device in the mouth, consists of a device formed by a nozzle or a smooth, corrugated, waved or cylindrical tubular element, which is inserted into the mouth and allows to produce a depression therein, either by extracting the smooth or cylindrical tubular element, or by sucking the air from the mouth with a corrugated tubular element by means of a flexible suction pump constituted by a bellows, bell or chamber in the shape of a deformable spherical cap. The smooth or cylindrical tubular element is inserted into the mouth and once it is adjusted to the lips, it is extracted, sucking much of the air, creating a depression that sucks the object that closes the throat. For greater effectiveness, these must have a larger diameter. The nozzle and the end of the corrugated tube are held with the teeth, sealing the lips the passage of air laterally and allowing it through the interior or central area. Except for the cylindrical device, which is solid or made of foam rubber, to prevent air from entering during use, the others have valves with a flexible tab which open when the device is inserted or pressed into the mouth and they close when the device or the suction pump is pulled. To hold the pump or the device, a handle, knob is used at the outermost end of the device, the latter attached, vertically or horizontally.

The system requires the obturation of the nose, pressing it with the fingers, or, for trained people, obturating or closing by means of the soft palate that blocks the nasopharynx, helped with the control of the muscles that act on the soft palate, or with the help of the tongue, blocking and sealing the nasopharyngeal area.

The materials used are rubber, thermoplastics, silicones or other polymers. Also thermoplastic rubber foams.

### BRIEF DESCRIPTION OF THE DRAWINGS

FIG. 1 shows a schematic, lateral and partial view of a device for extracting objects from the throat, of the smooth tubular type applied to a patient.

FIG. 2 shows a variant of the throat object extractor of conical shape.

FIG. 3 shows another variant of the throat object extractor of cylindrical shape

FIG. 4 shows another variant of the throat object extractor of cylindrical shape and having a nozzle

FIG. 5 shows another variant of the throat object extractor of cylindrical shape and having a nozzle to 6 shows another variant of the throat object extractor having a spherical cap shaped element.

FIG. 6 shows another variant of the throat object extractor having a spherical cap shaped element having a plate.

FIG. 7 shows a schematic and plan view of a nozzle used in the devices of FIGS. 4 to 6.

FIG. 8 shows a schematic and side view of the tubular type device used in FIG. 1.

FIG. 9 shows a view schematized and lateral view of the tubular type device used in FIG. 1, with a variant of the fastening or handle system.

FIG. 10 shows a schematic and side view of the tubular type device used in FIG. 1, with a variant of the fastening or handle system.

FIG. 11 shows a schematic and lateral view of a device of the corrugated tubular type, with a frustoconical shape, which acts as a bellows.

FIG. 12 shows a schematic and side view of a cylindrical device of the foam rubber type.

FIGS. 13 to 18 are taken from the magazine FASO, they roughly show the large number of elements that intervene in the area.

### MORE DETAILED DESCRIPTION OF AN EMBODIMENT FORM

FIG. 1 shows an embodiment of the system of the invention, with the extractor device (1), consisting of the tubular element (2), which is inserted and extracted using the handle (11v) and the support disk (4) at its outer end. The tubular element can be externally lubricated. The tubular element must have a large section, so that when it is extracted it tends to draw a large amount of air or to produce a depression in the oral cavity.

FIG. 2 shows the extractor device (1), which consists of the corrugated tubular element (3), and of conical and flexible shape, which acts as an extractor pump, with the cylindrical front end (3a) and consistent or reinforced, with channels that allows the introduction and clamping of the teeth, the outermost end being attached to the knob (5) which, being hollow, can carry the valve (6) that allows the air to escape when it is introduced and closes during its extraction.

FIG. 3 shows the extractor device (1), which consists of the corrugated tubular element (3), and of a cylindrical and flexible shape, which acts as an extractor pump, with the similarly cylindrical front end (3a) consistent or reinforced, with channels that allows the introduction and clamping of the teeth, the outermost end being attached to the knob (5) which, being hollow, can carry the valve (6) that allows the air to escape when it is introduced and closes during its extraction.

FIG. 4 shows the extractor device (1), which consists of the corrugated tubular element (3), and of cylindrical and flexible shape, which acts as an extractor pump, with the front end attached to the nozzle (7), of the type used to diving, with cavities that allow it to be held with the teeth, the outermost end being attached to the knob (5) which, being hollow, can carry the valve (6) that allows the air to escape when pressed and closes when pulled, allowing the extraction of air from the oral cavity.

FIG. 5 shows the extractor device (1), consisting of a spherical and flexible cap-shaped element (8), which acts as an extractor pump, with the front end attached to the mouthpiece (7), of the type used for diving, with cavities that allow it to be held with the teeth, the outermost end being attached to the handle (10), which can carry a valve, not shown in the figure, that allows the air to escape when it is introduced and closes during its extraction. The plate (14) acts as a support for the pump.

FIG. 6 shows the extractor device (1), which consists of the spherical and flexible cap-shaped element (8a) with the corrugated wall, which acts as an extractor pump, with the front end attached to the mouth nozzle (7), of the type used for diving, with cavities that allow it to be held with the teeth, the outermost end being attached to the handle (10), which can carry a valve, not shown in the figure, that allows the air to escape when it is introduced and closes during extraction. The plate (14) acts as a support for the pump.

FIG. 7 shows the mouth nozzle (7) used in the devices of FIGS. 4 to 6 with recesses or slots (9), in which the teeth are inserted to produce the device grip.

FIG. 8 shows the extractor device (1), with the tubular element (2), which is introduced and extracted by means of the handle (11v), carrying the support disk (4) at its outer end. The tubular element must have a large section, so that when it is extracted it tends to draw a large amount of air or to produce a depression in the oral cavity. The valve (6) allows the air to escape during its introduction and closes during the extraction of the device.

FIG. 9 shows the extractor device (1), with the tubular element (2), which is inserted and extracted by means of the handle (10), carrying the support disk (4) at its outer end. The tubular element must have a large section, so that when it is extracted it tends to draw a large amount of air or to produce a depression in the oral cavity. The valve (6) allows the air to escape during its introduction and closes during the extraction of the device.

FIG. 10 shows the extractor device (1), with the tubular element (2), which is inserted and extracted by means of the handle-puller (11), with the support disk (4) at its outer end. The tubular element must have a large section, so that when it is extracted it tends to draw a large amount of air or to produce a depression in the oral cavity. The valve (6) allows the air to escape during its introduction and closes during the extraction of the device.

FIG. 11 shows the extractor device (1), with the corrugated, flexible and frusto-conical tubular element (3), which acts as a bellows, with the outermost end attached to the knob (5) which, being hollow, can carry the valve (6) that allows air to escape when pressed and closes when pulled, allowing air to be extracted from the oral cavity. The ring (12) of soft material or foam rubber, at the innermost end of the buccal area, fits around the throat, closing the nasal air outlet.

FIG. 12 shows the extractor device (1) of the cylindrical type of foam rubber (12). Which carries the knob (5) at the outermost end. It may have a thin, lubricated outer shell. It must have a large diameter and its operation is similar to the tubular elements of FIGS. 8 to 10, but as it is not hollow, it does not use valves.

FIGS. 13 to 18 extracted from the journal FASO-Anatomy of the pharynx. By Drs. Juan Agustin Rodriguez D'Aquila (1), Matias Etcheverry (2), Guillermo Stipech (2). They show the large number of muscles, nerves, and other elements in the rhinopharynx, oropharynx, and laryngopharynx area. Therefore, since the functions of swallowing, breathing, etc. are involved and mixed. It is not difficult for problems to occur, even in healthy people, especially due to and increased by involuntary movements.

1. System for removing the objects from the throat, of the type that applies a suction device to the mouth, consists of a device formed by a cylindrical foam rubber element or a nozzle or a tubular element and a flexible suction pump made up of a bellows, deformable hemispherical chamber or bell.

2. System according to claim 1, wherein the tubular element is cylindrical and smooth.

3. System according to claim 1, wherein the tubular element is corrugated or waved.

4. System according to claim 1, wherein the suction pump has the shape of a bellows.

5. System according to claim 1, wherein the suction pump is shaped like deformable cap chamber or bell.

6. System according to claim 3, wherein the corrugated tubes have the innermost end reinforced and with channels where the teeth are placed to hold them.

7. System according to claim 1, wherein the nozzle has a depression or cavity to house the teeth and its fastening.

8. System according to claim 1, wherein the external end of said device comprises a knob or handle, the latter being able to be attached vertically or horizontally.

9. System according to claim 1, wherein the tubular elements or the hollow knobs or handles carry valves with flexible tabs.

10. System according to claim 1, wherein the materials used in the devices are rubber, thermoplastics, silicones and other polymers and thermoplastic foams.

11. System according to claim 3, wherein the corrugated tubular element (3) is flexible and cylindrical in shape.

12. System according to claim 3, wherein the corrugated tubular element (3) is flexible and frusto-conical in shape.

13. System according to claim 3, wherein the corrugated tubular element carries a ring (12) at its innermost end.
